(19) United States
(12) Patent Application Publication
(10) Pub. No.: US 2017/0333231 A1
(43) Pub. Date: Nov. 23, 2017

(54) MULTILAYERED EXPANDABLE VASCULAR STENT

(71) Applicant: DONGGUAN DIANFU PRODUCT DESIGN CO., LTD., Dongguan (CN)

(72) Inventor: Yangde LI, Dongguan (CN)

(21) Appl. No.: 15/671,096

(22) Filed: Aug. 7, 2017

Related U.S. Application Data
(63) Continuation-in-part of application No. PCT/CN2015/082153, filed on Jun. 24, 2015.

(30) Foreign Application Priority Data
Feb. 10, 2015 (CN) 201520095013.1

(51) Int. Cl. A61F 2/90; A61L 27/04; A61F 2/915

(52) U.S. Cl. CPC A61F 2/90; A61L 27/04; A61F 2002/9155

(57) ABSTRACT

A vascular stent including at least two layers of magnesium alloy latticed support walls, a cavity, and an expandable balloon. The expandable balloon is disposed in the cavity.

### CROSS-REFERENCE TO RELATED APPLICATIONS

This application is a continuation-in-part of International Patent Application No. PCT/CN2015/082153 with an international filing date of Jun. 24, 2015, designating the United States, now pending, and further claims priority to Chinese Patent Application No. 201520095013.1 filed Feb. 10, 2015. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND OF THE INVENTION

### Field of the Invention

The invention relates to the field of vascular stents, and more particularly to a multilayered expandable vascular stent.

### Description of the Related Art

In recent years, magnesium alloys have been widely used in clinical applications as cardiovascular stent materials. However, magnesium and magnesium alloy have poor corrosion resistance. In addition, the magnesium alloy stents implanted in cardiovascular systems tend to fracture or collapse under the impact of blood flow, posing severe adverse effects.

### SUMMARY OF THE INVENTION

In view of the above-described problems, it is one objective of the invention to provide a multilayered expandable vascular stent. Upon expansion of the vascular stent, the support walls in the outer layer have a relatively large expansion extent which exceeds the elongation percentage thereof, and tend to break or damage. The multilayered magnesium alloy latticed walls strengthen the support function of the stent, thus improving the security of the vascular stent.

To achieve the above objective, in accordance with one embodiment of the invention, there is provided a multilayered expandable vascular stent comprising at least two layers of magnesium alloy latticed support walls, a cavity, and an expandable balloon, the expandable balloon being disposed in the cavity.

In a class of this embodiment, the latticed support walls are between 0.05 and 0.2 mm in thickness.

In a class of this embodiment, surfaces of the latticed support walls are coated with a drug-loading layer. The drug-loading layer comprises a polymer and an active component. The polymer is an ethylene-vinyl alcohol copolymer, ethylene-vinyl acetate copolymer or styrene-isobutylene block copolymer. The active component is antiplatelet drug.

Advantages of the multilayered expandable vascular stent in accordance with embodiments of the invention are summarized as follows. The multilayered expandable vascular stent comprises at least two layers of latticed support walls, when the stent expands, the latticed support walls in the outer layer have a relatively large expansion extent which exceeds the elongation percentage thereof, and tend to break, while the latticed support walls in the inner layer have a relatively small expansion extent, and do not break, thus ensuring the integrity of the vascular stent, improving the security of the vascular stent, which is favorable to popularizing and using the vascular stent.

### BRIEF DESCRIPTION OF THE DRAWINGS

FIG. 1 is a schematic diagram of a multilayered expandable vascular stent in accordance with one embodiment of the invention;

FIG. 2 is a schematic diagram of a multilayered expandable vascular stent after being expanded in FIG. 1;

FIG. 3 is a schematic diagram of a multilayered expandable vascular stent in accordance with another embodiment of the invention; and

FIG. 4 is a schematic diagram of a multilayered expandable vascular stent after being expanded in FIG. 3.

### DETAILED DESCRIPTION OF THE EMBODIMENTS

For further illustrating the invention, experiments detailing a multilayered expandable vascular stent are described hereinbelow combined with the drawings. It should be noted that the following examples are intended to describe and not to limit the invention.

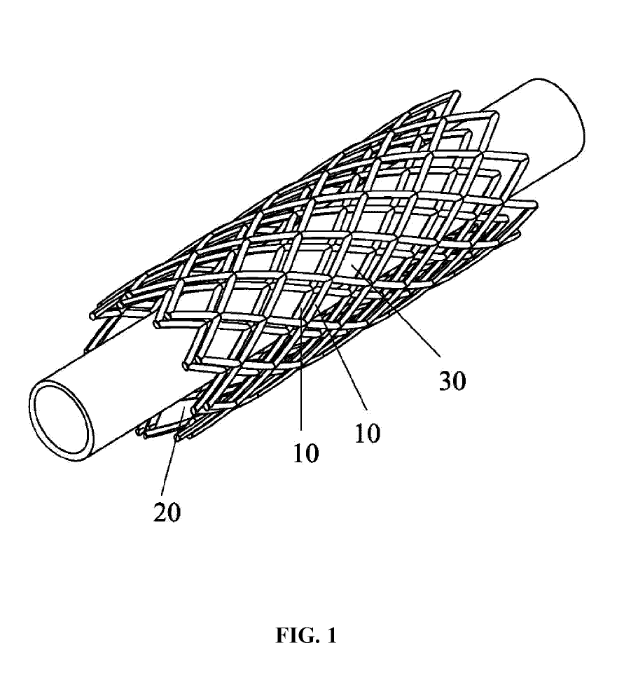

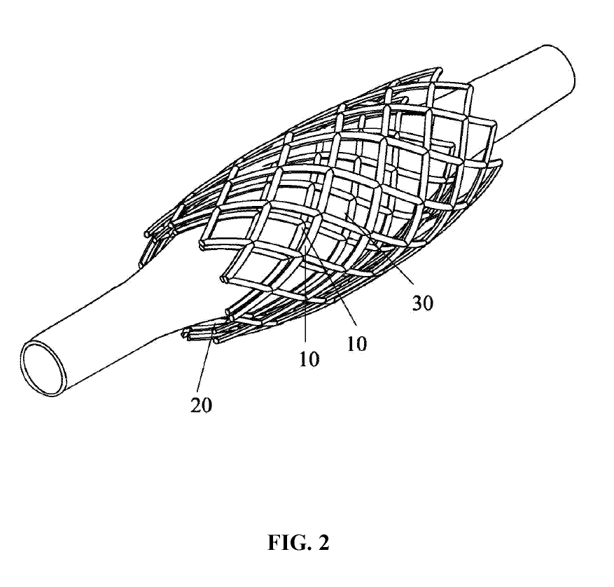

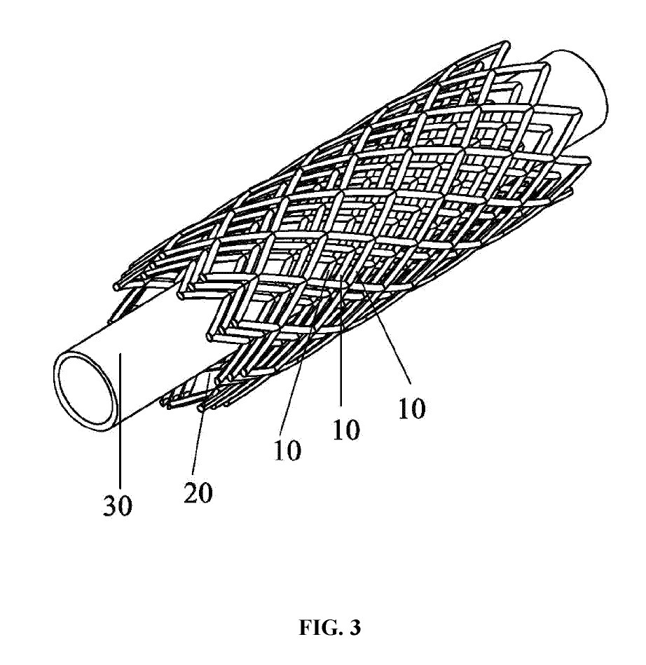

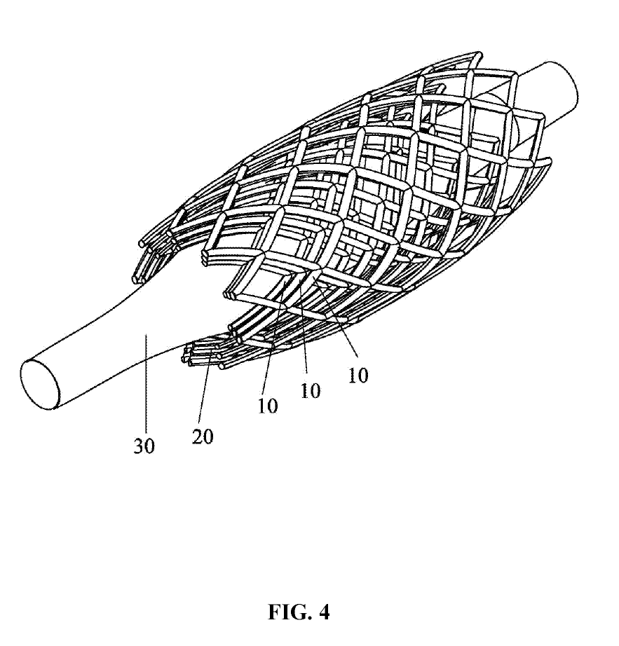

As shown in FIGS. 1, 2, 3, and 4, a multilayered expandable vascular stent comprises at least two layers of latticed support walls 10 and a cavity 20. An expandable balloon 30 is disposed in the cavity 20. The vascular vent is able to expand under the expansion of the balloon 30. The latticed support walls 10 in the outer layer have a relatively large expansion extent which exceeds the elongation percentage thereof, thus tend to break or damage, while the latticed support walls 10 in the inner layer have a relatively small expansion extent, and do not break and damage, thus ensuring the integrity of the vascular stent, and ensuring the vascular stent exhibits its role.

The latticed support walls are made of magnesium alloy material, which is degradable material and is degraded gradually with the time passing by. Thus, according to the requirement for service life, reasonable quantity of latticed support walls is preset. In general, there are at least two or three layers of latticed support walls. The latticed support walls in each layer are between 0.05 and 0.2 mm in thickness.

Preferably, surfaces of the latticed support walls are coated with a drug-loading layer. The drug-loading layer comprises a polymer and an active component. The polymer is an ethylene-vinyl alcohol copolymer, ethylene-vinyl acetate copolymer or styrene-isobutylene block copolymer. The active component is antiplatelet drug. The stent is arranged in the blood vessel to prevent the restenosis of the blood vessel thus achieving the purpose of dredging the blood vessel. So, it is necessary to coat antiplatelet drug on the blood vessel, for example, cilostazol, which not only has the function of antiplatelet, but also has the function of expanding blood vessel, inhibits the growth of vascular smooth muscle, and inhibits thrombosis, including vascular wall and endothelial cell.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A vascular stent, comprising:
at least two layers of magnesium alloy latticed support walls;
a cavity; and
an expandable balloon disposed in the cavity.

2. The stent of claim 1, wherein the latticed support walls are between 0.05 and 0.2 mm in thickness.

3. The stent of claim 1, wherein surfaces of the latticed support walls are coated with a drug-loading layer; the drug-loading layer comprises a polymer and an active component; the polymer is an ethylene-vinyl alcohol copolymer, ethylene-vinyl acetate copolymer or styrene-isobutylene block copolymer; and the active component is antiplatelet drug.
